(19) United States
(12) Patent Application Publication
(10) Pub. No.: US 2012/0264208 A1
(43) Pub. Date: Oct. 18, 2012

(54) MATERIALS AND METHODS FOR ENHANCED IRON UPTAKE IN CELL CULTURE

(76) Inventor: Sung-Wei Chen, Singapore (SG)

(21) Appl. No.: 12/995,656

(22) PCT Filed: Jul. 16, 2010
(86) PCT No.: PCT/SG10/00271

§ 371 Date: Dec. 1, 2010

(51) Int. Cl. C12N 5/071; C12N 5/0775; C12N 5/09; C12N 5/22

(52) U.S. Cl. 435/346; 435/375; 435/366; 435/369; 435/370; 435/367; 435/358

(57) ABSTRACT

A serum-free, synthetic tissue culture media is described which is completely defined chemically. The media do not require any supplementation with serum to support growth of cells. The media and methods described herein can also be used for growing all types of mammalian cell lines in culture without addition of transferrin protein. The media include a basal medium and an activator of iron uptake. The media also include a defined cell culture media that includes an iron-containing compound, which is capable of supporting the growth of mammalian cells in culture, increasing the level of expression of recombinant protein in cultured cells, and/or increasing virus production in cultured cells.

### TECHNICAL FIELD

The present technology relates generally to cell culture media and methods for mammalian cell culture.

### BACKGROUND

The following description is provided to assist the understanding of the reader. None of the information provided or references cited is admitted to be prior art.

Cells cultivated in culture media catabolize available nutrients while viable and especially during cell proliferation. Such cells may be useful in themselves, or as a means to produce a variety of useful biological substances, such as viruses, monoclonal antibodies, hormones, growth factors and the like. Such products have, for example, therapeutic applications and, with the advent of recombinant DNA technology, cells can be engineered to produce large quantities of many of these products. Mammalian cell culture is used in many recombinant protein production processes due to its ability to produce proteins with proper post-translational modifications. Thus, the ability to cultivate cells in vitro is not only important for the study of cell physiology, but is also necessary for the production of cells useful substances which may not otherwise be obtained using cost-effective production.

Cell culture media formulations have been well documented in the literature and a number of media are commercially available. Cell culture media provide the nutrients necessary to maintain and grow cells in a controlled, artificial environment. Characteristics and compositions of the cell culture media vary depending on the particular cellular requirements. Important parameters include osmolarity, pH, and nutrient formulations. The requirements of mammalian cell culture in vitro include, in addition to basic nutritional substances, a complex array of growth factors. Usually, these are added to the culture medium by supplying it with animal sera or protein fractions from animal sources. However, these chemically undefined mixtures exhibit lot to lot variability. Such mixtures also represent a potential source of contaminants, including viruses and mycoplasmas. For production on an industrial scale, the high price of the supplements and difficulties in downstream processing are additional considerations.

Growth, metabolism, and maintenance of cells requires iron as an essential nutrient. Most mammalian cell culture systems use transferrin (Tf), a serum protein, as a primary, staple iron source/transporter. It is so indispensable for these culture systems that it is frequently referred to as a “growth factor.” Eukaryotic transferrins comprise a class of bilobal iron-binding proteins, each lobe bearing a single site capable of reversibly binding iron and accounting for the physiological roles of the proteins in iron transport and iron withholding. Tf normally provides iron for cellular needs, and for most cells the delivery of transferrin-borne iron depends on association of the protein with transferrin receptors, TfR1 and TfR2, on plasma membranes. An elaborate receptor-mediated pathway drives endocytosis of Tf-bound iron into mammalian cells for use and storage. TfR1 and TfR2 play critical roles in iron transfer involving transferrin. Transferrin is often obtained from animal-derived serum—causing sourcing, contamination, and quality assurance problems, among many others—or through transgenic production—making it an expensive additive.

### SUMMARY

In one aspect, the present disclosure provides a method for enhancing iron uptake in a mammalian cell culture, the method comprising: contacting cells with a first culture medium containing an effective amount of an activator of iron uptake; replacing the first culture medium with a second culture medium containing a source of iron; and incubating the cells under conditions suitable to allow the growth of the cells in culture.

In one embodiment, the activator is a multivalent ion. In one embodiment, the multivalent ion is selected from the group consisting of: Fe³⁺, Ga³⁺, Gd³⁺, Al³⁺, La³⁺, Zr⁴⁺, Sn⁴⁺, Cu²⁺, and Zn²⁺. In one embodiment, the activator is in the form of an ionic salt, selected from the group consisting of: nitrates, nitriles, citrates, sulfates, sulfides, halides, nitrites, organic salts, and hydrated salts. In one embodiment, the activator is ferric ammonium citrate (FAC). In one embodiment, the FAC is present in the first culture medium in a final concentration of at least 100 ng/mL. In one embodiment, the FAC is present in the first culture medium in a final concentration of about 100 ng/mL to about 100 μg/mL. In one embodiment, the activator is Ga(NO₃)₃. In one embodiment, the activator is a mitogen. In one embodiment, wherein the mitogen is selected from the group consisting of: phytohemagglutinin, concanavalin A (conA), lipopolysaccharide (LPS), or pokeweed mitogen (PWM).

In one embodiment, the first culture medium lacks inhibitors of induction. In one embodiment, the inhibitors of induction are selected from the group consisting of Ca²⁺ and free radical scavengers. In one embodiment, the free radical scavengers are selected from the group consisting of: catalase, superoxide dismutase, and mannitol.

In one embodiment, the source of iron is an iron-organic ion chelate. In one embodiment, the iron-organic ion chelate is ferric ammonium citrate (FAC). In one embodiment, the FAC is present in the second culture medium in a final concentration of at least 100 ng/mL. In one embodiment, the FAC is present in the second culture medium in a final concentration of about 100 ng/mL to about 100 μg/mL.

In one embodiment, the cells are human cells or human hybrid cells. In one embodiment, the human cells are selected from the group consisting of: lyphocytes, myeloid cells, monocytes, macrophages, neutrophils, myocytes, fibroblasts, HepG2 carcinoma cells, kidney cells, melanoma cells, and HeLa cells. In one embodiment, the cells are non-human mammalian cells. In one embodiment, the non-human mammalian cells are Chinese hamster ovary cells.

In one embodiment, the cells are contacted with the first culture medium for from about 15 minutes to about 1 hour. In one embodiment, the cells are contacted with the first culture media for about 30 minutes. In one embodiment, both the first culture medium and the second culture medium lack transferrin. In one embodiment, both the first culture medium and the second culture media are serum-free media. In one embodiment, the cells are rinsed prior to being contacted with the first culture medium. In one embodiment, the cells are rinsed after being contacted with the first culture medium. In one embodiment, the steps of contacting and replacing occur in a cell reactor.

In another aspect, the present disclosure provides a kit for enhancing iron uptake in mammalian cell culture comprising a first culture medium additive containing an activator of iron uptake; and a second culture medium additive containing a source of iron, wherein both the first culture medium additive and second culture medium additive lack transferrin.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 is flow diagram showing a sequence of steps carried out in accordance with an illustrative embodiment.

### DETAILED DESCRIPTION

In the following detailed description, reference may be made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here. In the description that follows, a number of terms are used extensively. The terms described below are more fully understood by reference to the specification as a whole. Units, prefixes, and symbols may be denoted in their accepted SI form.

The terms “a” and “an” as used herein mean “one or more” unless the singular is expressly specified. Thus, for example, reference to “a cell” includes a mixture of two or more cells, as well as a single cell.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “activator of iron uptake” refers to a compound that activates a non-transferrin bound iron (NTBI) transport pathway. In some embodiments, the activator of iron uptake is a multivalent ion that may be in the form of an ionic salt. In illustrative embodiments, the multivalent ion is Fe³⁺, Ga³⁺, Gd³⁺, Al³⁺, La³⁺, Zr⁴⁺, Sn⁴⁺, Cu²⁺, and/or Zn²⁺. In another embodiment, the activator of iron uptake is a mitogen, such as phytohemagglutinin.

As used herein, the term “cytokine” refers to a compound that induces a physiological response in a cell, such as growth, differentiation, senescence, apoptosis, cytotoxicity or antibody secretion. Included in this definition of “cytokine” are growth factors, interleukins, colony-stimulating factors, interferons, lymphokines and the like.

As used herein, the term “cell culture” or “culture” is meant the maintenance, growth, and proliferation of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” or “organ system culture” may occasionally be used interchangeably with the term “cell culture.” The media described herein can be used to culture any mammalian cell.

As used herein, the term “cultivation” is meant the maintenance of cells in vitro under conditions favoring growth, differentiation or continued viability, in an active or quiescent state, of the cells. In this sense, “cultivation” may be used interchangeably with “cell culture” or any of its synonyms described above.

As used herein, the term “culture vessel” is meant a glass, plastic, or metal container that can provide an aseptic environment for culturing cells.

As used herein, the term phrases “cell culture medium,” “culture medium” (plural “media” in each case) and “medium formulation” refer to a nutritive solution for cultivating cells and may be used interchangeably.

As used herein, the term “contacting” refers to the placing of cells to be cultivated in vitro into a culture vessel with the medium in which the cells are to be cultivated. The term “contacting” encompasses mixing cells with medium, pipetting medium onto cells in a culture vessel, and submerging cells in culture medium.

As used herein, the term “combining” refers to the mixing or admixing of ingredients in a cell culture medium formulation.

As used herein, a “chemically defined” medium is one for which every ingredient is known. A chemically defined medium is distinguished from serum, embryonic extracts, and hydrolysates, each of which contain unknown components.

As used herein, the term “ingredient” refers to any compound, whether of chemical or biological origin, that can be used in cell culture media, to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein, a “protein-free” medium is one which contains no proteins or peptides. A protein-free medium is distinguished from low-protein and essentially protein-free media, both of which contain proteins and/or peptides.

The term “transport,” as in the “transport” of a compound of interest across a cell membrane refers to passage of the compound in the direction of external to internal movement.

The terms “optional” and “optionally” mean that the subsequently described circumstance may or may not occur, so that the description includes instances where the circumstance occurs and instances where it does not.

### Cell Culture Media

The present disclosure provides cell culture media and methods that use activators of non-transferrin bound iron (NTBI) uptake in mammalian cells. The culture media and methods may also enhance overall iron transfer into cells as part of a serum free cell culture system. The present disclosure provides, inter alia, methods for increasing iron uptake in a mammalian cell culture using transferrin-dependent and non-transferrin-dependent mechanisms. As such, these methods allow for the production of economical serum-free and/or protein-free media for cell culture.

Typically, cell culture media formulations are supplemented with a range of additives, including undefined components such as fetal bovine serum (FBS) or extracts from animal embryos, organs or glands. While FBS is the most commonly used supplement in animal cell culture media, other serum sources are also routinely used, including newborn calf, horse and human. These types of chemically undefined supplements serve several useful functions in cell culture media. For example, these supplements provide carriers or chelators for labile or water-insoluble nutrients; bind and neutralize toxic moieties; provide hormones and growth factors, protease inhibitors and essential, often unidentified or undefined low molecular weight nutrients; and protect cells from physical stress and damage. Thus, serum extracts are commonly used as supplements to provide an optimal culture medium for the cultivation of mammalian cells.

Unfortunately, the use of serum or protein additives in tissue culture applications has several drawbacks. For example, the chemical compositions of these supplements and sera vary between lots, even from a single manufacturer. The supplements may also be contaminated with infectious agents (e.g., mycoplasma and viruses) which can seriously undermine the health of the cultured cells and the quality of the final product. The use of undefined components such as serum or animal extracts also prevents the true definition and elucidation of the nutritional and hormonal requirements of the cultured cells, thus eliminating the ability to study, in a controlled way, the effect of specific growth factors or nutrients on cell growth and differentiation in culture. Finally, serum and protein supplementation of culture media can complicate and increase the costs of the purification of the desired substances from the culture media due to nonspecific co-purification of serum or extract proteins.

To overcome these drawbacks of the use of serum or organ/gland extracts, the present disclosure provides media that are specifically formulated to use a non-transferrin bound iron (NTBI) transport pathway. NTBI pathway(s) import iron through low molecular weight chelators, such as citrates, nitrates, or sulfates. As such, chemically defined media can be used that do not rely on the addition of serum or recombinant transferrin.

In one aspect, the present disclosure relates to culture media and methods for cultivating a mammalian cell in vitro. In one embodiment, the methods include replacing protein (particularly animal-derived or recombinant transferrin) in mammalian cell culture media with chemically-defined mixtures. In particular, the disclosure relates to replacing transferrin, to media containing such replacements, and to compositions comprising mammalian cells in such media. In one embodiment, the present disclosure relates to a first culture medium containing an activator of iron uptake and a second culture medium containing source of iron. The present technology also relates to media for suspension culture and to compositions comprising mammalian cells in such suspension culture. Improved levels of recombinant protein expression may be obtained from cells treated with an activator of iron uptake, relative to the level of expression seen in cells grown in medium supplemented with serum.

These culture media and methods allow the replacement of or reduction in the amount of transferrin compared to conventional mammalian culture media, with all the associated advantages. Contrasted with naturally derived (e.g. animal serum) transferrin, this lowers or eliminates dangers of contamination. Likewise, compared to recombinant transferrin, the present compositions and methods lowers or eliminates the cost for using the recombinant protein. These methods also allow for granular and dynamic control of iron uptake, since process parameters (e.g. amount of inductor, incubation time, temperature, etc.) may be finely controlled.

In some embodiments, cells are incubated transiently with the activator of iron uptake by charging of cells with a medium containing the activator of iron uptake. Induction by the activators causes the cells to endogenously increase their uptake of iron. In one embodiment, the serum-free cell culture medium includes one or more activators of iron uptake. The one or more activators of iron uptake may be a multivalent ion, e.g., Fe³⁺, Ga³⁺, Gd³⁺, Al³⁺, La³⁺, Zr⁴⁺, Sn⁴⁺, Cu²⁺, and/or Zn²⁺. In some embodiments, the multivalent ion may take the form of an ionic salt, e.g., nitrates, nitriles, citrates, sulfates, and/or sulfides containing the multivalent ion. In one embodiment, the ionic salt of the multivalent ion is ferric ammonium citrate (FAC). In one embodiment, the FAC is present in the first culture medium in a final concentration of at least 100 ng/mL. In some embodiments, the ionic salts of the multivalent ions are added at a final concentration of about 100 ng/mL to about 100 μg/mL.

In one embodiment, Zn²⁺-containing compounds may be used, including but are not limited to, citrates, chlorides, halides, nitrates, nitrites, nitriles, sulfides, sulfates, organic salts, and/or hydrated salts, such as ZnCl, Zn(NO₃)₂, ZnBr, and ZnSO₄.7H₂O. In some embodiments, the ionic salts of the multivalent Zn²⁺-containing compounds are added at a final concentration of about 100 ng/mL to about 100 μg/mL.

In another embodiment, the activator of iron uptake is a mitogen. A mitogen is a chemical substance that encourages a cell to commence cell division, triggering mitosis. Mitogens trigger signal transduction pathways in which mitogen-activated protein kinase is involved, leading to mitosis. In an illustrative embodiment, the mitogen is phytohemagglutinin (See Sturm. B. et al., “The Influence of gallium and Other Metal Ions on the Uptake of Non_Transferrin Bound Iron by Rat Hepatocytes,” Biochimie (2006) 88: 645-650). In other embodiments, the mitogen is concanavalin A (conA), lipopolysaccharide (LPS), or pokeweed mitogen (PWM).

In some embodiments, cells are incubated in a culture medium containing a source of iron after they have been contacted with an activator of iron uptake. In one embodiment, the source of iron is a Fe²⁺ and/or Fe³⁺ chelate compound. Illustrative Fe²⁺ and/or Fe³⁺ salts and chelators include ethylenediaminetetraacetic acid (EDTA), ethylene glycol-bis(β-aminoethyl ether)-N,N,N′,N′-tetraacetic acid (EGTA), deferoxamine mesylate, dimercaptopropanol, diethylenetriaminepentaacetic acid (DPTA), ferric ammonium citrate (FAC) and trans-1,2-diaminocyclohexane-N,N,N.N-tetraacetic acid (CDTA). For example, the iron chelate compound may be a ferric citrate chelate, such as ferrous ammonium citrate. In one embodiment, the iron chelate compound used is ferrous sulphate 7H₂O EDTA (FeSO₄.7H₂O.EDTA, e.g., Sigma F0518. Sigma, St. Louis, Mo.). In some embodiments, the concentration of Fe²⁺ and/or Fe³⁺ in the medium can be about 100 ng/mL to about 100 μg/mL.

The culture media may further include one or more ingredients selected from the group of ingredients consisting of one or more amino acids, one or more vitamins, one or more inorganic salts, one or more sugars, one or more buffering salts, and one or more lipids. In one embodiment, the sugar used in the media is D-glucose, while the buffer salt may be N-[2-hydroxyethyl]-piperazine-N′-[2-ethanesulfonic acid] (HEPES). In one embodiment, the culture media may optionally comprise one or more supplements selected from the group of supplements consisting of one or more cytokines, heparin, one or more animal peptides, one or more yeast peptides and one or more plant peptides.

The amino acid ingredients of the present media may include one or more amino acids selected from the group consisting of L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. The vitamin ingredient of the present media may include one or more vitamins selected from the group consisting of biotin, choline chloride, D-Ca²⁺-pantothenate, folic acid, i-inositol, niacinamide, pyridoxine, riboflavin, thiamine and vitamin B₁₂. The inorganic salt ingredient of the present media may include one or more inorganic salts selected from the group consisting of one or more calcium salts, Fe(NO₃)₃, KCl, one or more magnesium salts, one or more manganese salts, NaCl, NaHCO₃, Na₂HPO₄, one or more selenium salts, one or more vanadium salts and one or more zinc salts.

The media may also include the ingredients ethanolamine, D-glucose, N-[2-hydroxyethyl]piperazine-N′-[2-ethanesulfonic acid] (HEPES), insulin, linoleic acid, lipoic acid, phenol red, PLURONIC F68, putrescine, sodium pyruvate, biotin, choline chloride, D-Ca²⁺-pantothenate, folic acid, i-inositol, niacinamide, pyridoxine, riboflavin, thiamine, vitamin B₁₂, one or more calcium salts, Fe(NO₃)₃, KCl, one or more magnesium salts, one or more manganese salts, NaCl, NaHCO₃, Na₂HPO₄, one or more selenium salts, one or more vanadium salts and one or more zinc salts, wherein each ingredient is present in an amount which supports the cultivation of a mammalian cell in vitro.

The specific combinations of the above ingredients and their concentration ranges, in one example of the culture media containing an activator of iron uptake are shown in Table 1.

TABLE 1
Illustrative Cell Culture Media Composition Containing Activator
of Iron Uptake

| Component | Illustrative Embodiment (mg/L) |
|---|---|
| Ferric ammonium citrate | 0.075 |
| (Iron uptake activator) | |
| Glycine | 7.5 |
| L-Alanine | 8.9 |
| L-Arginine hydrochloride | 211 |
| L-Asparagine-H₂O | 15.01 |
| L-Aspartic acid | 13.3 |
| L-Cysteine hydrochloride-H₂O | 35.12 |
| L-Glutamic Acid | 14.7 |
| L-Glutamine | 146 |
| L-Histidine hydrochloride-H₂O | 21 |
| L-Isoleucine | 4 |
| L-Leucine | 13.1 |
| L-Lysine hydrochloride | 36.5 |
| L-Methionine | 4.5 |
| L-Phenylalanine | 5 |
| L-Proline | 34.5 |
| L-Serine | 10.5 |
| L-Threonine | 11.9 |
| L-Tryptophan | 2.04 |
| L-Tyrosine disodium salt dihydrate | 7.81 |
| L-Valine | 11.7 |
| Biotin | 0.0073 |
| Choline chloride | 14 |
| D-Calcium pantothenate | 0.5 |
| Folic Acid | 1.3 |
| Niacinamide | 0.036 |
| Pyridoxine hydrochloride | 0.06 |
| Riboflavin | 0.037 |
| Thiamine hydrochloride | 0.3 |
| Vitamin B12 | 1.4 |
| i-Inositol | 18 |
| Calcium Chloride (CaCl₂) (anhyd.) | 33.22 |
| Cupric sulfate (CuSO₄—5H₂O) | 0.0025 |
| Magnesium Chloride (anhydrous) | 57.72 |
| Potassium Chloride (KCl) | 223.6 |
| Sodium Bicarbonate (NaHCO₃) | 1176 |
| Sodium Chloride (NaCl) | 7599 |
| Sodium Phosphate dibasic (Na₂HPO₄) | 142 |
| anhydrous | |
| D-Glucose (Dextrose) | 1802 |
| Hypoxanthine Na | 4.77 |
| Linoleic Acid | 0.084 |
| Lipoic Acid | 0.21 |
| Phenol Red | 1.2 |
| Putrescine 2HCl | 0.161 |
| Sodium Pyruvate | 110 |
| Thymidine | 0.7 |

The specific combination of the above ingredients and their concentration ranges, in one example of a culture media containing a source of iron is shown in Table 2.

TABLE 2
Illustrative Cell Culture Media Composition Containing Iron Source

| Component | Illustrative Embodiment (mg/L) |
|---|---|
| Ferric sulfate (FeSO₄—7H₂O) | 0.05 |
| Glycine | 7.5 |
| L-Alanine | 8.9 |
| L-Arginine hydrochloride | 211 |
| L-Asparagine-H₂O | 15.01 |
| L-Aspartic acid | 13.3 |
| L-Cysteine hydrochloride-H₂O | 35.12 |
| L-Glutamic Acid | 14.7 |
| L-Glutamine | 146 |
| L-Histidine hydrochloride-H₂O | 21 |
| L-Isoleucine | 4 |
| L-Leucine | 13.1 |
| L-Lysine hydrochloride | 36.5 |
| L-Methionine | 4.5 |
| L-Phenylalanine | 5 |
| L-Proline | 34.5 |
| L-Serine | 10.5 |
| L-Threonine | 11.9 |
| L-Tryptophan | 2.04 |
| L-Tyrosine disodium salt dihydrate | 7.81 |
| L-Valine | 11.7 |
| Biotin | 0.0073 |
| Choline chloride | 14 |
| D-Calcium pantothenate | 0.5 |
| Folic Acid | 1.3 |
| Niacinamide | 0.036 |
| Pyridoxine hydrochloride | 0.06 |
| Riboflavin | 0.037 |
| Thiamine hydrochloride | 0.3 |
| Vitamin B12 | 1.4 |
| i-Inositol | 18 |
| Calcium Chloride (CaCl₂) (anhyd.) | 33.22 |
| Cupric sulfate (CuSO₄—5H₂O) | 0.0025 |
| Magnesium Chloride (anhydrous) | 57.22 |
| Potassium Chloride (KCl) | 223.6 |
| Sodium Bicarbonate (NaHCO₃) | 1176 |
| Sodium Chloride (NaCl) | 7599 |
| Sodium Phosphate dibasic (Na₂HPO₄) | 142 |
| anhydrous | |
| D-Glucose (Dextrose) | 1802 |
| Hypoxanthine Na | 4.77 |
| Linoleic Acid | 0.084 |
| Lipoic Acid | 0.21 |
| Phenol Red | 1.2 |
| Putrescine 2HCl | 0.161 |
| Sodium Pyruvate | 110 |
| Thymidine | 0.7 |

As will be readily apparent to one of ordinary skill in the art, the concentration of a given ingredient can be increased or decreased beyond the range disclosed and the effect of the increased or decreased concentration can be determined using only routine experimentation. The optimal final concentrations for medium ingredients for culturing particular cell types are typically identified either by empirical studies, in single component titration studies. In single component titration studies using animal cells (e.g., CHO cells or 293 embryonic kidney cells), the concentration of a single medium component is varied while all other constituents and variables are kept constant and the effect of the single component on viability, growth or continued health of the animal cells is measured.

Medium ingredients can be dissolved in a liquid carrier or maintained in dry form. The type of liquid carrier and the method used to dissolve the ingredients into solution vary and may include periodic or continuous mixing, stirring, or shaking, optionally including heating to assist in dissolving the ingredients. In one embodiment, the liquid carrier is water. In another embodiment, the liquid carrier is a buffer, e.g., HEPES or MOPS buffer. In yet another embodiment, the liquid carrier is a concentrated medium lacking one or more components, e.g., an activator of iron uptake and/or an iron source. In one embodiment, the pH of the medium is adjusted to about 7.0-7.6. about 7.1-7.5, or about 7.2-7.4. In one embodiment, the osmolarity of the medium is adjusted to about 260 to about 300 mOsm, about 265 to about 280 mOsm, or about 265 to about 275 mOsm. The type of liquid carrier and the method used to dissolve the ingredients into solution vary and can be determined by one of ordinary skill in the art with no more than routine experimentation. Typically, the medium ingredients can be added in any order.

In some embodiments, the solutions comprising individual ingredients are more concentrated than the concentration of the same ingredients in a 1× media formulation. The ingredients can be 10-fold more concentrated (10× formulation), 25-fold more concentrated (25× formulation), 50-fold more concentrated (50× concentration), or 100-fold more concentrated (100× formulation). More highly concentrated formulations can be made, provided that the ingredients remain soluble and stable.

If the individual medium ingredients are prepared as separate concentrated solutions, an appropriate (sufficient) amount of each concentrate is combined with a diluent to produce a 1× medium formulation. Typically, the diluent used is water but other solutions including aqueous buffers, aqueous saline solution, or other aqueous solutions may be used.

The culture media are typically sterilized to prevent unwanted contamination. Sterilization may be accomplished, for example, by filtration through a low protein-binding membrane filter of about 0.22 μm or 0.45 μm pore size (available commercially, for example, from Millipore, Bedford, Mass.) after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation. Autoclaving or other elevated temperature-based methods of sterilization are not favored, since many of the components of the present culture media are heat labile and will be irreversibly degraded by temperatures such as those achieved during most heat sterilization methods.

### Use of Culture Media and Methods

In one aspect, the cell culture media may be used to facilitate cultivation of a variety of mammalian cells in suspension or in monolayer cultures. In particular, these media may be used to cultivate mammalian cells or cell lines. Methods for isolation, and suspension and monolayer cultivation, of a variety of animal cells including mammalian cells are known in the art (see, e.g., Freshney, R. I., Culture of Animal Cells: A Manual of Basic Technique, New York: Alan R. Liss, Inc. (1983)) and are described in further detail. While the present media are particularly useful for culturing mammalian cells in suspension, it is to be understood that the media may be used in any standard cell culture protocol whether the cells are grown in suspension, in monolayers, in perfusion cultures (e.g., in hollow fiber microtube perfusion systems), on semi-permeable supports (e.g., filter membranes), in complex multicellular arrays or in any other method by which mammalian cells may be cultivated in vitro.

The media and methods disclosed herein may be used to culture a variety of mammalian cells, including primary epithelial cells (e.g., keratinocytes, cervical epithelial cells, bronchial epithelial cells, tracheal epithelial cells, kidney epithelial cells and retinal epithelial cells) and established cell lines (e.g., 293 embryonic kidney cells, HeLa cervical epithelial cells and PER-C6 retinal cells, MDBK (NBL-1) cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, I-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestine, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃HJIOTI/2 cells, HSDM₁C₃ cells, KLN205 cells, McCoy cells, Mouse L cells, Strain 2071 (Mouse L) cells, L-M strain (Mouse L) cells, L-MTK.sup.-(Mouse L) cells, NCTC clones 2472 and 2555, SCC-PSA1 cells, Swiss/3T3 cells, Indian muntjac cells, SIRC cells, C.sub.II cells, and Jensen cells, or derivatives thereof).

Cells supported by the medium may be derived from any animal, such as, but not limited to, a mammal. In one embodiment, the cells are derived from a human. In one embodiment, the cells are mammalian epithelial or fibroblast cells. In illustrative embodiments, the cells are 293 embryonic kidney cells, PER-C6 retinal cells, or CHO cells. The cells cultivated in the present media may be normal cells or abnormal cells (i.e., transformed cells, established cells, or cells derived from diseased tissue samples).

Animal cells for culturing in the media may be obtained commercially, for example from ATCC (Rockville, Md.), Quantum Biotechnologies (Montreal, Canada) or Invitrogen (San Diego, Calif.). Alternatively, cells may be isolated directly from samples of animal tissue obtained via biopsy, autopsy, donation or other surgical or medical procedure.

The present disclosure provides methods of cultivating cells using the culture medium formulations disclosed herein, comprising contacting cells with a first culture medium containing an effective amount of an activator of iron uptake; replacing the first culture medium with a second culture medium containing a source of iron; and incubating the cells under conditions suitable to allow the growth of the cells in culture. In one embodiment, the cells are incubated in a medium containing an activator of iron uptake for at least 30 minutes, at least 60 minutes, at least 2 hours, at least 4 hours, at least 6 hours, at least 8 hours, at least 12 hours, at least 24 hours, or at least 48 hours. In some embodiments, the cells are not incubated in a medium containing an activator of iron uptake for longer than about 48 hours or longer than about 24 hours.

Optionally, the cells are rinsed before and/or after being contacted with the activator of iron uptake. In one embodiment, the cells are then placed into a media containing a source of iron. The iron may be present in the form of low molecular weight chelates. Optionally, if one seeks to utilize both transferrin-dependent and non-transferrin dependent iron uptake mechanisms, then recombinant or animal-derived transferrin may be added to the media. In this embodiment, the activators of iron uptake are used to enhance or augment the iron transport capability of the cell culture. Transferrin may be in the iron-free form (i.e., apotransferrin) or in the iron-complexed form (i.e., ferrotransferrin or holotansferrin).

The present disclosure further relates to methods of cultivating mammalian cells (particularly those described above) in suspension comprising (a) obtaining a mammalian cell to be cultivated in suspension; (b) contacting the cell with a culture medium including an activator of iron uptake under conditions sufficient to activate an NTBI transport pathway; and (c) transferring the cell to a culture medium that includes a source of iron.

The present methods further relate producing a polypeptide, and to polypeptides produced by these methods, the methods comprising (a) obtaining a mammalian cell, such as a 293 embryonic kidney epithelial cell, PER-C6, and CHO cell, that has been genetically engineered to produce a polypeptide; and (b) contacting the cell with a culture medium including an activator of iron uptake under conditions sufficient to activate an NTBI transport pathway; and (c) transferring the cell to a culture medium that includes a source of iron. For example, the polypeptide may be any polypeptide of interest for research or therapeutic purposes.

Optimal methods for genetically engineering a mammalian cell to express a polypeptide of interest are well-known in the art and will therefore be familiar to one of ordinary skill. Cells may be genetically engineered prior to cultivation in the media described herein, or they may be transfected with one or more exogenous nucleic acid molecules after being placed into culture in the media. According to one embodiment, genetically engineered cells may be cultivated in the present culture media either as monolayer cultures, or as suspension cultures according to the methods described above. Following cultivation of the cells, the polypeptide of interest may optionally be purified from the cells and/or the used culture medium according to techniques of protein isolation that will be familiar to one of ordinary skill in the art.

The present disclosure further relates to methods of producing a virus, and to viruses produced by these methods, the methods comprising (a) obtaining a mammalian cell, such as a 293 embryonic kidney epithelial cell, PER-C6, or CHO cell, to be infected with a virus; (b) contacting the cell with a virus under conditions suitable to promote the infection of the cell by the virus; (c) contacting the cell with a culture medium including an activator of iron uptake under conditions sufficient to activate an NTBI transport pathway; and (d) transferring the cell to a culture medium that includes a source of iron. Viruses which may be produced according to these methods include adenoviruses, adeno-associated viruses and retroviruses.

In various embodiments, the cell may be contacted with the virus either prior to, during or following cultivation of the cell in the culture media disclosed herein; optimal methods for infecting a mammalian cell with a virus are known. Virus-infected mammalian cells cultivated in suspension in the media may be expected to produce higher virus titers (e.g., 2-, 3-, 5-, 10-, 20-, 25-, 50-, 100-, 250-, 500-, or 1000-fold higher titers) than those cells not cultivated in suspension in the media. These methods may be used to produce a variety of mammalian viruses and viral vectors, including but not limited to adenoviruses, adeno-associated viruses, retroviruses and the like, and are used to produce adenoviruses or adeno-associated viruses. Following cultivation of the infected cells in the present media, the used culture media comprising viruses, viral vectors, viral particles or components thereof (proteins and/or nucleic acids (DNA and/or RNA)) may be used for a variety of purposes, including vaccine production, production of viral vectors for use in cell transfection or gene therapy, infection of animals or cell cultures, study of viral proteins and/or nucleic acids and the like. Alternatively, viruses, viral vectors, viral particles or components thereof may optionally be isolated from the used culture medium according to techniques for protein and/or nucleic acid isolation that will be familiar to one of ordinary skill in the art.

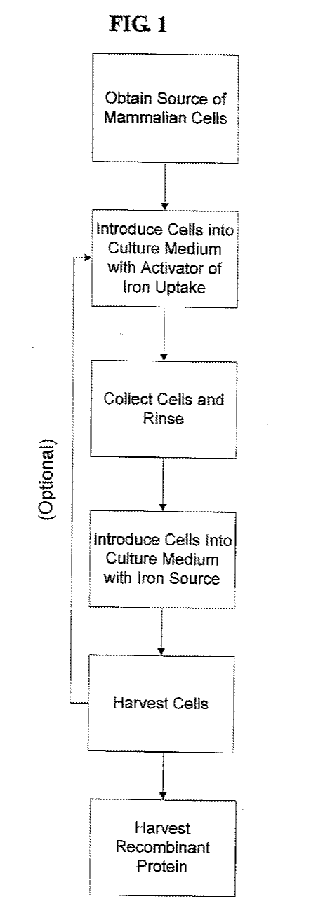

FIG. 1 shows an illustrative embodiment of the culture methods. A source of mammalian cells, e.g., CHO cells, are obtained and added to a culture medium containing an activator of iron uptake (e.g., FAC). The cells are incubated for a period of time sufficient to activate an NTBI pathway, e.g. for at least 5 min, at least 10 min, at least 30 min, at least 1 hour, at least 2 hours, at least 4 hours, but typically not more than about 8 hours or not more than about 1 or 2 days. Next, the cells are removed from the first culture medium by e.g., decanting or centrifugation, and added to a second culture medium containing a source of iron. The cells are incubated for a period of time to expand the cells and/or produce a recombinant protein. Next, the cells and/or recombinant protein are harvested. Optionally, an aliquot of the cells may be reintroduced into a first medium containing an activator of iron uptake, and the process is repeated. Optionally, the second culture medium may be omitted and the first culture medium may be sufficient to induce the cells as well as to provide the source of iron. In some embodiments, cells in closed or batch culture undergo complete medium exchange (i.e., replacing spent media with fresh media) when the cells reach a density of about 1.5-2.0×10⁶ cells/ml. Cells in perfusion culture (e.g., in bioreactors or fermenters) will receive fresh media on a continuously recirculating basis.

The cell seeding densities can be optimized for the specific culture conditions being used. For routine monolayer culture in plastic culture vessels, an initial seeding density of 1-5×10⁵ cells/cm² may be used, while for suspension cultivation a higher seeding density (e.g., 5-20×10⁵ cells/cm²) may be used.

Mammalian cells are typically cultivated in a cell incubator at about 37° C. The incubator atmosphere should be humidified and should contain about 3-10% carbon dioxide in air, about 8-10% carbon dioxide in air, or about 8% carbon dioxide in air, although cultivation of certain cell lines may require as much as 20% carbon dioxide in air for optimal results.

### Kits

The disclosure also provides kits for use in the cultivation of a mammalian cells. Kits comprise one or more containers, wherein a first container contains the first culture medium, including an activator of iron uptake; and a second container containing the second culture medium including a source of iron. These kits may farther comprise one or more additional containers containing one or more supplements.

Additional kits may include one or more containers wherein a first container contains a basal culture medium prepared as described above and a second container contains an activator of iron uptake. The media in the containers of these kits may be present as dry powders, 1× ready-to-use formulations, or as more concentrated solutions (for example 2×, 5×, 10×, 20×, 25×, 50×, 100×, 500×, 1000× or higher). Additional kits may further comprise one or more additional containers containing one or more supplements selected from the group consisting of one or more cytokines, heparin, one or more peptides, etc.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of will be understood to include those elements specifically recited and those additional elements that do not materially affect the basic and novel characteristics of the claimed technology. The phrase “consisting of excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths. etc. As a non-limiting example, each range discussed herein can be readily broken down into a lower third, middle third and upper third. etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for enhancing iron uptake in a mammalian cell culture, the method comprising:
contacting cells with a first culture medium containing an effective amount of an activator of iron uptake;
replacing the first culture medium with a second culture medium containing source of iron; and
incubating the cells under conditions suitable to allow the growth of the cells in culture.

2. The method of claim 1, wherein the activator is a multivalent ion.

3. The method of claim 2, wherein the multivalent ion is selected from the group consisting of Fe³⁺, Ga³⁺, Gd³⁺, Al³⁺, La3+, Zr⁴⁺, Sn⁴⁺, Cu²⁺, and Zn²⁺.

4. The method of claim 3, wherein the activator is ferric ammonium citrate (FAC).

5. The method of claim 4, wherein the FAC is present in the first culture medium in a final concentration of at least 100 ng/mL.

6. The method of claim 4, wherein the FAC is present in the first culture medium in a final concentration of about 100 ng/mL to about 100 μg/mL.

7. The method of claim 3, wherein the activator is in the form of an ionic salt, selected from the group consisting of: nitrates, nitriles, citrates, sulfates, sulfides, halides, nitrites, organic salts, and hydrated salts.

8. The method of claim 3, wherein the activator is Ga(NO₃)₃.

9. The method of claim 1, wherein the activator is a mitogen.

10. The method of claim 9, wherein the mitogen is selected from the group consisting of: phytohemagglutinin, concanavalin A (conA), lipopolysaccharide (LPS), or pokeweed mitogen (PWM).

11. The method of claim any of claims 1-10, wherein the first culture medium lacks inhibitors of induction.

12. The method of claim 11, wherein the inhibitors of induction are selected from the group consisting of Ca²⁺ and free radical scavengers.

13. The method of claim 12, wherein the free radical scavengers are selected from the group consisting of catalase, superoxide dismutase, and mannitol.

14. The method of any of claims 1-13, wherein the source of iron is an iron-organic ion chelate.

15. The method of claim 14, wherein the iron-organic ion chelate is ferric ammonium citrate (FAC).

16. The method of claim 15, wherein the FAC is present in the second culture medium in a final concentration of at least 100 ng/mL.

17. The method of claim 15, wherein the FAC is present in the second culture medium in a final concentration of about 100 ng/mL to about 100 μg/mL.

18. The method of any of claims 1-17, wherein the cells are human cells or human hybrid cells.

19. The method of claim 18, wherein the human cells are selected from the group consisting of: lyphocytes, myeloid cells, monocytes, macrophages, neutrophils, myocytes, fibroblasts, HepG2 carcinoma cells, kidney cells, melanoma cells, and HeLa cells.

20. The method of claim any of claims 1-17, wherein the cells are non-human mammalian cells.

21. The method of claim 20, wherein the non-human mammalian cells are Chinese hamster ovary cells.

22. The method of claim any of claims 1-21, wherein the cells are contacted with the first culture medium for from about 15 minutes to about 1 hour.

23. The method of claim 22, wherein the cells are contacted with to first culture media for about 30 minutes.

24. The method of claim any of claims 1-23, wherein both the first culture medium an the second culture medium lack transferrin.

25. The method of claim any of claims 1-24, wherein both the first culture medium and the second culture media are serum-free media.

26. The method of claim any of claims 1-25, wherein the cells are rinsed prior to being contacted with the first culture medium.

27. The method of claim any of claims 1-26, wherein the cells are rinsed after being contacted with the first culture medium.

28. The method of claim any of claims 1-27, wherein the steps of contacting and replacing occur in a cell reactor.

29. A kit for enhancing iron uptake in mammalian cell culture comprising:
a first culture medium additive containing an activator of iron uptake; and
a second culture medium additive containing a source of iron, wherein both the first culture medium additive and second culture medium additive lack transferrin.
